(19) United States
(12) Patent Application Publication
(10) Pub. No.: US 2020/0289311 A1
(43) Pub. Date: Sep. 17, 2020

(54) ORAL APPLIANCE

(71) Applicant: Elias Albert ALFI, Tarzana, CA (US)

(72) Inventor: Elias Albert ALFI, Tarzana, CA (US)

(21) Appl. No.: 16/790,006

(22) Filed: Feb. 13, 2020

Related U.S. Application Data
(60) Provisional application No. 62/916,454, filed on Oct. 17, 2019.
(60) Provisional application No. 62/816,859, filed on Mar. 11, 2019.

(51) Int. Cl. A61F 5/56; A61C 5/90

(52) U.S. Cl. CPC A61F 5/566; A61C 5/90

(57) ABSTRACT

The present disclosure relates to an oral appliance adapted to be worn over a user's teeth comprising an upper guard and lower guard that are configured to prevent or mitigate the effects of tongue thrusting and improper tongue positioning, particularly during sleep.

### CROSS-REFERENCE TO RELATED APPLICATIONS

This application claims priority benefit of U.S. provisional patent applications 62/816,859, filed Mar. 11, 2019, and 62/916,454, filed Oct. 17, 2019, each of which is hereby incorporated by reference herein.

### FIELD

The present disclosure relates to an oral appliance, in particular an oral appliance adapted to be worn over a user's teeth comprising an upper guard and lower guard; and methods of using the oral appliance for treating disorders associated with improper tongue positioning.

### BACKGROUND

Tongue thrusting (also called reverse swallow or immature swallow) occurs when the tongue is pushed forward between the upper and lower front teeth during swallowing, instead of pushing against the gum above the back of the upper front teeth. Tongue thrusting can adversely affect the teeth and mouth by forcing the teeth out of alignment. Dental appliances that retrain the tongue have met with some success however tongue thrust often returns when use of the appliance is discontinued. That is, if the tongue is allowed to continue pushing against the teeth, it will reverse the orthodontic work.

Treatment for uncontrolled tongue thrusting (e.g., during sleep) typically involves habit retraining in the form of physical therapy for the tongue and/or the use of a dental appliance. Known dental appliances for treating tongue thrust have employed prongs and spikes with mixed results. Patient discomfort is a common complaint and can result in the patient discontinuing use of the dental appliance. Temporary effectiveness is another common complaint. Accordingly, there exists a need for an oral appliance that is comfortable yet effective in minimizing the adverse impact of tongue thrusting on teeth, especially for a user who experiences tongue-thrusting during sleep.

Additionally, improper positioning of the tongue has a direct relation to numerous sleep disorders, including sleep panda. Indeed, in most cases, following Maxillomandibular Advancement surgery a patient's sleep apnea problem often is cured due to the ability of the tongue to move into a position further forward during sleep. Accordingly, there exists a need for an oral appliance that can properly position a user's tongue during sleep, and thereby can be used to treat or prevent sleep disorders (including sleep apnea).

### SUMMARY

As well as the embodiments discussed in the summary, other embodiments are disclosed in the specification, drawings, and claims. The summary is not meant to cover each and every embodiment; combination or variations are contemplated with the present disclosure.

In some embodiments, the present disclosure provides an oral appliance to be worn in a user's mouth, wherein the oral appliance comprises an upper guard and a lower guard that are configured to provide an upper ramp and a lower ramp that act to guide the user's tongue through a gap between the user's upper and lower front teeth when the tongue is thrusted forward. The oral appliance configuration thereby minimizes or prevents pressure exerted by the tip of the user's tongue against a lingual surface of the upper and/or lower front teeth that may cause misalignment of the upper and lower front teeth. The oral appliance structure comprising upper and lower ramps also prevents the user's tongue from situating its tip immediately behind the upper and lower front teeth and being able to push against the lingual surface of the upper and lower front teeth to cause misalignment. Generally, the oral appliance can worn by a user at any time when prevention of tongue-thrusting or improper tongue positioning is desired, especially during sleeping. In some embodiments, the oral appliance comprises upper and lower guards having risers (or “raised formations”) between the user's molars, wherein the risers act to keep the user's teeth from fully occluding which allows a gap to exist between the user's upper and lower front teeth. When the tongue is thrusted forward, the ascending ramp of the lower guard and the descending ramp of the upper guard work together to direct the tip of the tongue to move past the upper and lower front teeth and protrude from the gap. As such, the tip of the tongue cannot rest behind the upper and lower front teeth to exert pressure against the lingual surface of the upper or lower front teeth to cause misalignment.

In some embodiments, the oral appliance has an upper guard and a lower guard, wherein the upper guard has a lower surface that is ramped with a downward slope from a posterior end of the upper guard to an anterior end of the upper guard, and the lower guard has an upper surface that is ramped with an upward slope from a posterior end of the lower guard to an anterior end of the lower guard. As described elsewhere herein, the respective downward and upward ramped configurations of the upper and lower guards of the oral appliance acts as a chute that guides the user's tongue to extend between the upper and lower front teeth and away from their lingual surfaces. In some embodiments, the oral appliance further comprises a means for immobilizing the tongue in an extended position between the upper and lower front teeth and away from their lingual surfaces.

In some embodiments, the oral appliance has an upper guard and a lower guard, wherein the upper guard has a lower surface that is devoid of any lingual surface that is substantially vertical, and the lower guard has an upper surface that is devoid of any lingual surface that is substantially vertical.

In some embodiments, the oral appliance has an upper guard and a lower guard, wherein the upper guard has a lower surface that is devoid of any surface that is substantially parallel to a lingual surface of a user's front upper teeth; and the lower guard has an upper surface that is devoid of any surface that is substantially parallel to a lingual surface of a user's front lower teeth.

In some embodiments, the oral appliance has an upper guard and a lower guard, wherein: the upper guard has a posterior end, an anterior end, an upper surface, a lower surface, and a recess formed in the upper surface configured to receive a user's upper teeth; and the lower guard has a posterior end, an anterior end, an upper surface, a lower surface, and a recess formed in the lower surface configured to receive the user's lower teeth. In some embodiments, the lower surface of the upper guard is configured without a surface that is generally parallel to a lingual surface of the recess configured to receive the user's upper teeth, and the upper surface of the lower guard is configured without a surface generally parallel to a lingual surface of the recess configured to receive the user's lower teeth. The absence of such a surface generally parallel to a lingual surface in either the lower surface of the upper guard or the upper surface of the lower guard, the oral appliance provides no surface against which a user's tongue can thrust, and allows for the user's tongue to extend forward between the users' teeth.

In some embodiments, the first lower surface of the upper guard has a generally continuous downward slope from the first posterior end to the first anterior end. In some embodiments, the second upper surface of the lower guard has a generally continuous upward slope from the second posterior end to the second anterior end.

In some embodiments, the upper guard has a descending ramp and the lower guard as an ascending ramp, wherein the descending and the ascending ramps are configured to guide the tongue toward a gap between anterior ends of the upper and lower guards.

In some embodiments, the upper guard has a generally semi-circular configuration when viewed from above. In some embodiments, the upper guard has a generally larger wedge-shaped cross-section when viewed from the side.

In some embodiments, the upper guard has a thinner posterior end, a thicker anterior end, and a mid-portion with increasing thickness from the posterior end to the anterior end.

In some embodiments, the lower guard has a generally “C” configuration when viewed from above. In some embodiments, the lower guard has a generally smaller wedge-shaped cross-section when viewed from the side.

In some embodiments, the lower guard has a thinner posterior end, a thicker anterior end, and a mid-portion with increasing thickness from the posterior end to the anterior end.

In some embodiments, the lower surface of the upper guard below a portion of the first curved recess configured to receive the user's upper molars has a riser (or “raised formation”) configured to prevent full occlusion by the user. In some embodiments, the upper surface of the lower guard above a portion of the second curved recess configured to receive the user's lower molars has a riser to prevent full occlusion by the user.

In some embodiments, the upper guard and the lower guard are detached from each other. In some embodiments, the upper guard and the lower guard are attached at one or more locations at or near their respective posterior ends; optionally, wherein the attachment locations are flexible or hinged.

In some embodiments, the present disclosure also provides for uses and methods for treating or preventing disorders associated with improper tongue positioning, such as tongue thrusting or sleep disorders (e.g., sleep apnea), wherein the methods comprise using an oral appliance of the present disclosure, whereby the user's tongue extends in a forward position, optionally, wherein the extended forward position comprises a position between the upper and lower front teeth and away from their lingual surfaces. In embodiments for treating or preventing a sleep disorder, the uses and methods comprise using an oral appliance of the present disclosure during sleep to maintain a user's tongue in an extended position, optionally, wherein the extended position comprises a position between the upper and lower front teeth and away from their lingual surfaces.

### BRIEF DESCRIPTION OF THE DRAWINGS

Some embodiments can be understood in more detail from the following description taken in conjunction with the accompanying drawings, in which:

FIG. 1 is a top perspective view of an upper guard of an oral appliance in accordance with an embodiment of the present invention.

FIG. 2 is a bottom perspective view of the upper guard of FIG. 1.

FIG. 3 is a top perspective view of a lower guard of an oral appliance in accordance with an embodiment of the present invention.

FIG. 4 is a bottom perspective view of the lower guard of FIG. 3.

FIG. 5 is a side cross-sectional view of the upper and bottom guards of FIGS. 1 and 3, as used in a person's mouth, taken along a center longitudinal axis.

FIG. 6 is a plan view looking upwardly at the upper guard as worn on a person's upper teeth.

FIG. 7 is a plan view looking downwardly at the lower guard as worn on a person's lower teeth.

FIG. 8 is a side view of a joined oral appliance with hinged upper and lower guards.

FIG. 9 is a side, upwardly perspective view of the oral appliance of FIG. 8.

FIG. 10 is a side cross-sectional view of an upper guard and a lower guard that leaves at least buccal surfaces of upper and lower front teeth exposed.

FIG. 11 is a side cross-sectional view of the upper and bottom guards of an oral appliance in a user's mouth showing the tongue in an extended position between the upper and lower front teeth.

### DETAILED DESCRIPTION

Features of the inventive concept and methods of accomplishing the same may be understood more readily by reference to the following detailed description of embodiments and the accompanying drawings. Hereinafter, where embodiments are described with reference to the accompanying drawings. Unless otherwise noted, like reference numerals denote like elements throughout the attached drawings and the written description, and thus, descriptions thereof will not be repeated. The drawings are not necessarily to scale and the relative sizes of elements, layers, and regions shown may be exaggerated for clarity. The present disclosure should not be construed as being limited to only the illustrated embodiments herein. Rather, the illustrated embodiments provided herein are intended as examples that convey aspects and features of the present disclosure to those skilled in the art. Processes, elements, and techniques that are not necessary to those having ordinary skill in the art for a complete understanding of the aspects and features of the present disclosure may not be described.

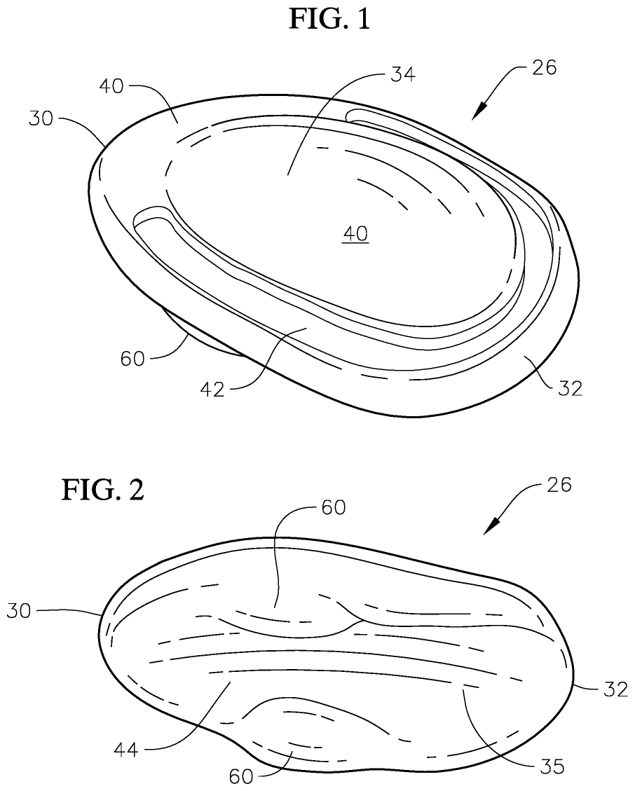

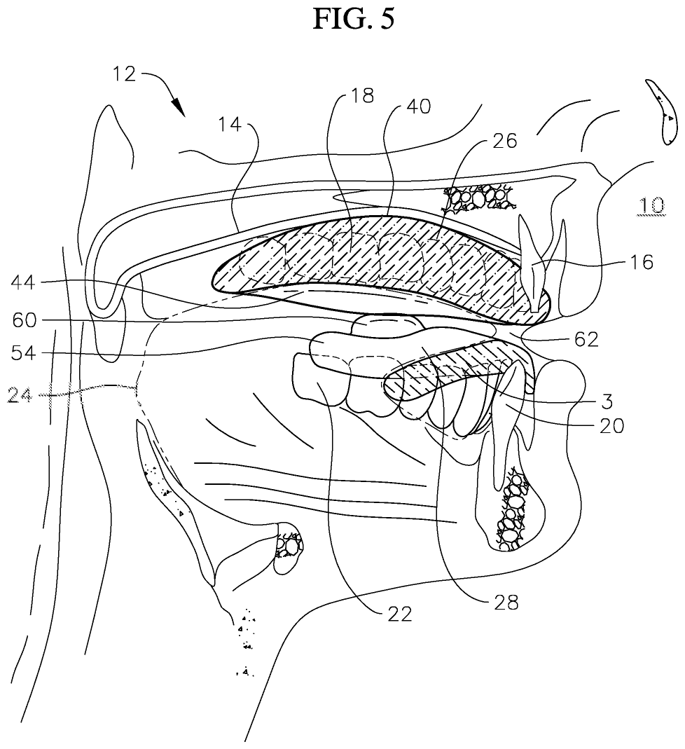

Embodiments of the oral appliance of the present disclosure are shown in FIGS. 1-11. As illustrated in FIGS. 5, 10, and 11 the oral appliance 10 is designed for use inside a user's mouth 12 having a palate or roof 14, teeth (including upper front teeth 16 and upper molars 18, lower front teeth 20 and lower molars 22), and a tongue 24. The oral appliance has an upper guard 26, as shown in FIGS. 1, 2, and 6, and a lower guard 28, as shown in FIGS. 3, 4, and 7.

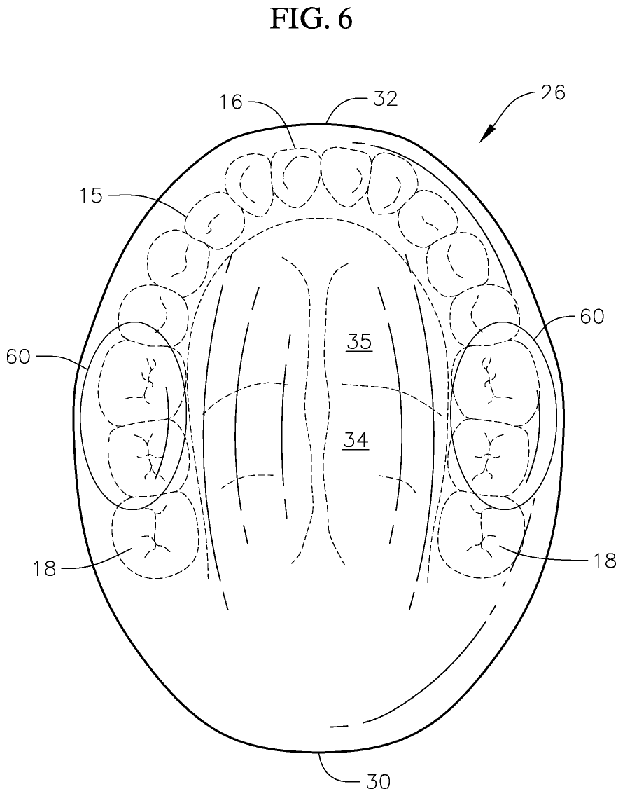

The upper guard 26 has a solid body and a lower surface 44 that is advantageously configured without any lingual surface that is generally vertical or generally parallel to a lingual surface of the first recess. In some embodiments, the upper guard 26 has a generally wedge cross-section with a thinner posterior end 30, a relatively thicker anterior end 32, and a mid-portion 34 that progressively thickens from the posterior end 30 to the anterior end 32. When viewed from above, as shown in FIGS. 1 and 6, the upper guard anterior end 32 has a generally semi-circular shape that follows the curvature of the user's upper teeth 15 and the posterior end 30 (which may be slightly convex, generally linear or a slight concave, as desired or appropriate) between the user's upper molars to provide a more comfortable profile against the user's palate 14 and tongue 24. Along the anterior end 32, upper surface 40 of the upper guard 26 has a curved recess or indentation 42 that is configured to receive the user's upper teeth 15, including the upper front teeth 16 and the upper molars 18. In the mid-portion 34 circumscribed by the curved recess 42, the upper surface 40 of the upper guard 26 facing the palate 14 is generally convex so as to closely follow the concavity of the palate 14.

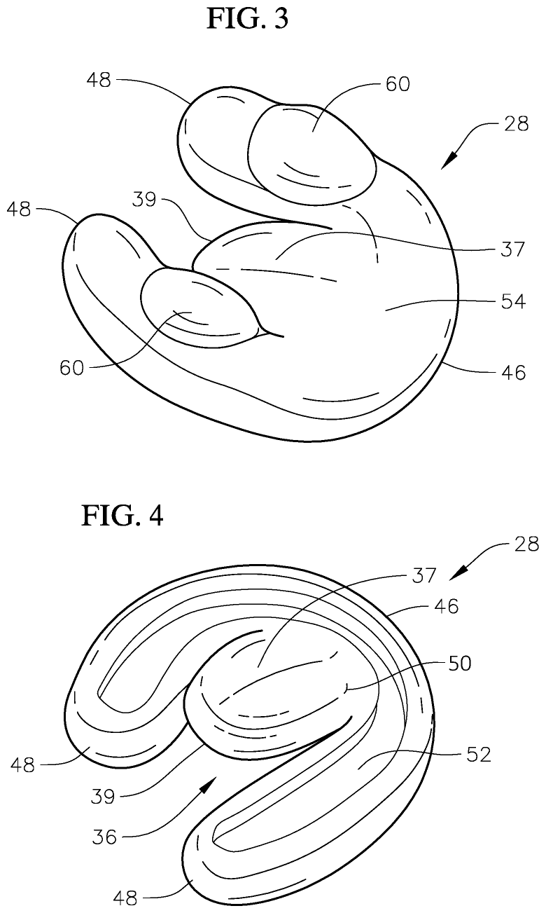

As shown in FIG. 2, the lower surface 44 of the upper guard 26 facing the user's tongue 24 has a slight concavity so as to closely follow the slightly convexity of an upper surface of the tongue when it is relaxed. The upper guard 26 has a wedge shape (when viewed from the side) due to a generally continuous thickening of the upper guard between the upper surface 40 and the lower surface 44 from the posterior end 30 of the upper guard toward the anterior end 32 of the upper guard. The thickening is especially pronounced near the front region of the curved recess 42 adapted to receive the user's upper front teeth 16. In that front region of the upper guard 26, the lower surface 44 remains below the maximum depth of the curved recess 42 at least in the front region that receives the user's upper front teeth 16. Thus, the lower surface 44 of the upper guard 26 is advantageously without (or devoid of) any generally vertical surface behind the user's upper front teeth 16. That is, the lower surface of the upper guard is devoid of any surface that is generally parallel with the lingual surface (LSU) of the curved recess 42 for the upper front teeth 16. As such, the lower surface 44 of the upper guard 26 is sloped downwardly with a descending ramp 35 toward the upper front teeth 16 from the posterior edge 30 to the anterior edge 32 such that the upper guard 26 is without any surface against which the front of the tongue 24 can effectively thrust on or press against to exert a force on the lingual side of the upper front teeth 16. The lower surface 44 is therefore without any surface against which the tip of the tongue can effectively rest behind the upper front teeth and thrust against to cause misalignment of the upper front teeth.

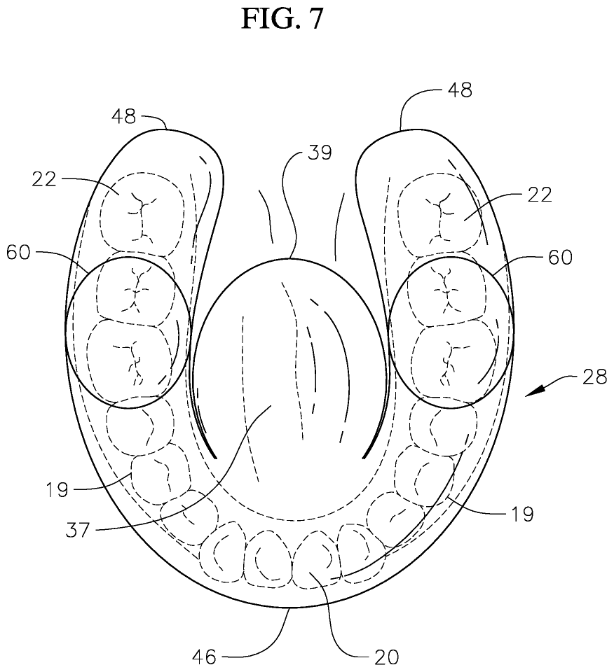

Viewed from the top, as shown in FIGS. 4 and 7, the lower guard 28 has a body resembling a “C” shape with a curved anterior end 46 and left and right posterior ends 48 that follow the relatively larger curvature of the user's lower teeth 19, including lower front teeth 20 and lower molars 22, while providing an inset 36 that accommodates the base of the tongue. A lower surface 50 of the lower guard has a curved recess or indentation 52 that is configured to receive the user's lower teeth 19. At the inset 36 of the “C” shaped configuration, an ascending ramp 37 is provided with an upper surface 54 that is sloped upwardly from a relatively thinner posterior end 39 to the relatively thicker anterior end 46. An upper surface 54 of the lower guard 28 is advantageously configured without any lingual surface that is generally vertical, or any surface that is generally parallel to a lingual surface of the curved recess 52. An upper surface 54 of the ramp 37 is therefore without any surface against which the tip of the user's tongue 24 can effectively rest behind the lower front teeth and thrust against to cause misalignment.

In some embodiment, the upper surface 54 in the front region of the lower guard 28 remains above the maximum height of the curved recess 52 in the front region that receives the user's lower front teeth 20. The upper surface 54 of the lower guard, thus, is advantageously without (or devoid of) any generally vertical surface behind the user's lower front teeth 20 that is generally parallel with a lingual surface (LSL) of the curved recess 52 for the lower front teeth 20. As such, the upper surface 54 of the lower guard 28 is sloped upwardly toward the lower front teeth 20 from the posterior end 39 to the anterior end 46 such that the lower guard 28 is without any surface against which the front of the tongue 24 can effectively thrust on or press against to exert a force on the lingual side LSL of the lower front teeth 20.

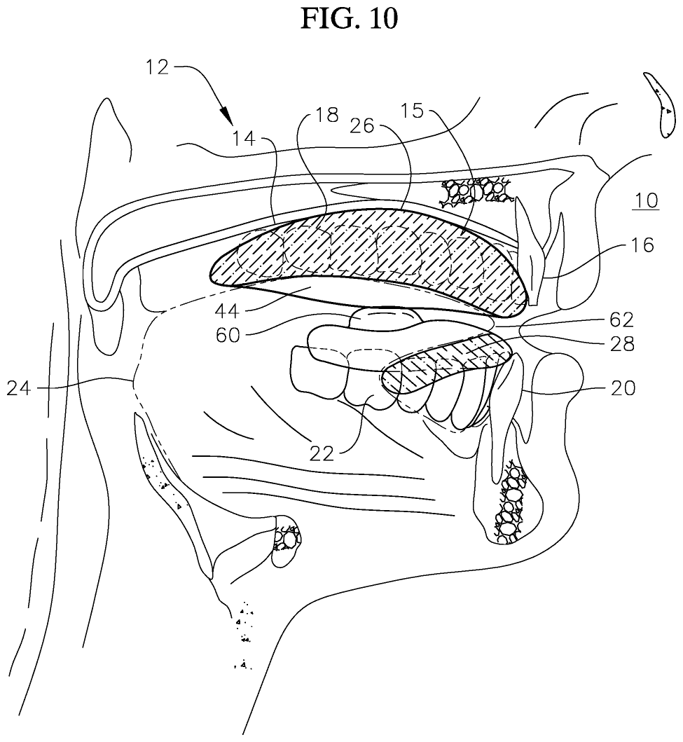

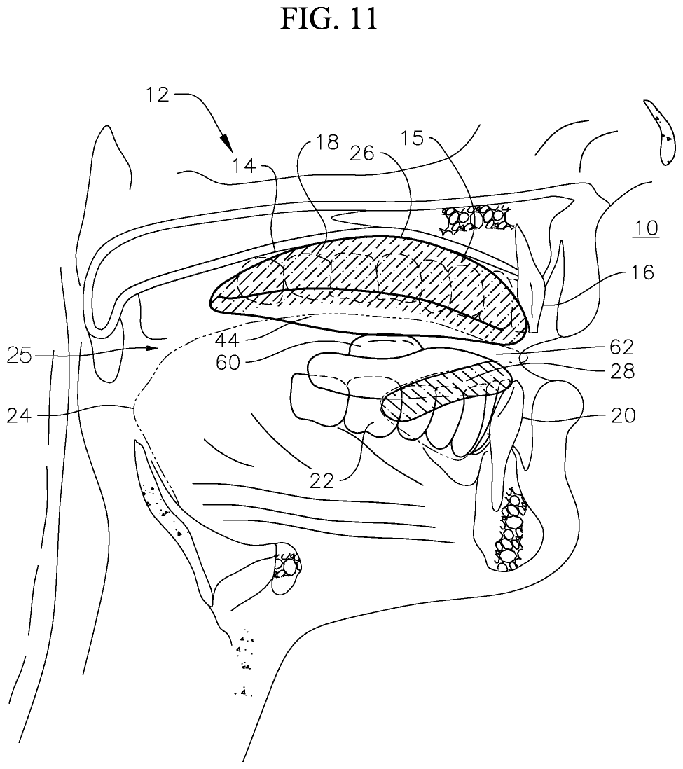

In some embodiments, the lower surface 46 of the upper guard 26 below the upper right and left molars 18, and/or the upper surface 54 of the lower guard 28 above the lower right and left molars 22 include one or more risers 60 (e.g., raised formations, raised mounds, or portions of sufficient thickness), as shown in FIGS. 2 and 3. These risers 60 may oppose each other and come in contact when the user bites down thereby preventing the user from completely occluding his teeth. As such, the risers 60 allow a gap 62 (see FIGS. 5 and 10) to exist between the upper guard 26 and the lower guard 28 especially near the user's front teeth 16 and 20. The existence of the gap 62 allows the tip of the user's tongue 24 to protrude between the front of the upper and lower guards 26 and 28. With the tongue 24 situated between the lower surface 44 of the upper guard 26 and the upper surface 54 of the lower guard 28, if the user thrusts his tongue forward the lower and upper surfaces 44, 54 guide the tongue forward into the gap 62 thus preventing the tongue from resting or being able to exert significant force against the lingual sides LSU, LSL of the user's upper and lower front teeth 16, 20.

It is understood that the risers 60 are optional and that the oral appliance may or may not include the risers. Even without the risers, the oral appliance is configured to naturally allow the user's bite to open when the tongue is thrust forward because the ramping of the lower surface 44 of the upper guard 26 and the upper surface 54 of the lower guard 28 causes the tip of the user's tongue moving forward to separate the user's upper and lower front teeth and open the user's bite.

In some embodiments, the upper guard 26 is configured such that buccal surface (BSU) of the upper front teeth 16 is outside of the guard 26 and thus exposed, whereas the posterior upper teeth such as the upper molars 18 are received in left and right recesses formed in the upper surface 40 of the upper guard 26 to anchor the upper guard 26. The thicker or “ramped” anterior end 32 of the upper guard 26 therefore rests against the lingual surface of the upper front teeth 16, with the lower surface 44 of the upper guard 26 having a downward slope from the posterior end 30 to the anterior end 32 such no lingual surface of the upper front teeth is exposed and the lower surface 44 is without any vertical surface that is generally parallel to the lingual surface LSU of the upper front teeth 16.

In some embodiments, the lower guard 28 is configured such that buccal surface (BSL) of the lower front teeth 20 is outside of the guard 28 and thus exposed, whereas the posterior lower teeth such as the lower molars 22 are received in left and right recesses formed in the lower surface 50 of the lower guard 28 to anchor the lower guard 28. The thicker or “ramped” anterior end 46 of the upper guard 28 therefore rests against the lingual surface of the lower front teeth 20, with the upper surface 54 of the lower guard 28 having an upward slope from the posterior end 48 to the anterior end 46 such no lingual surface of the lower front teeth is exposed and the upper surface 54 is without any vertical surface that is generally parallel to the lingual surface LSL of the lower front teeth 20.

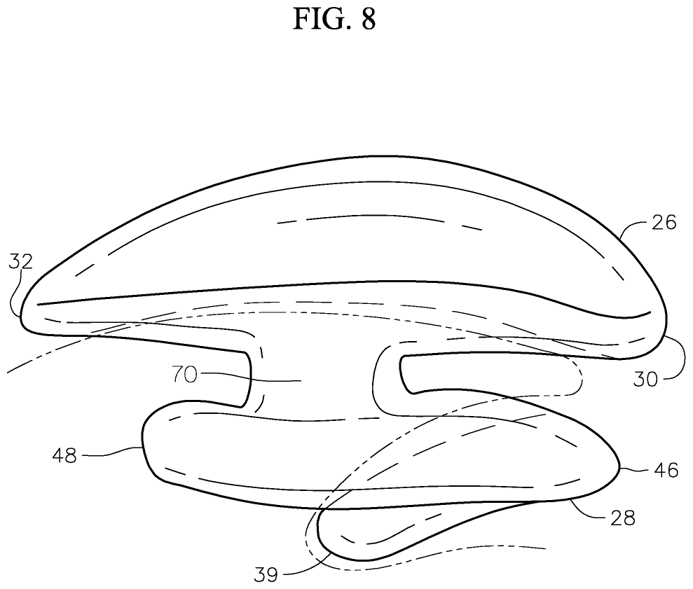

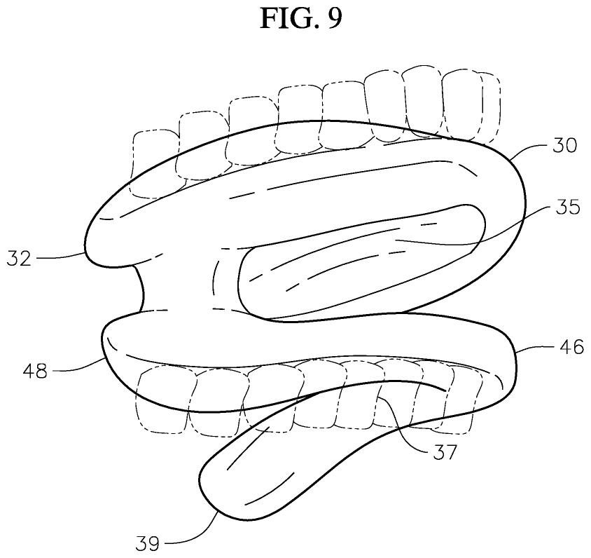

In some embodiments of the oral appliance, the upper and lower guards are separate members. It is also contemplated that in some embodiments of the oral appliance, the upper and lower guards are attached forming a single member, or single unit. Accordingly, as shown in FIGS. 8 and 9, in some embodiments, the upper guard 26 and lower guard 28 are attached at one or more locations 70 at or near their posterior ends. In some embodiments, the attachment locations are flexible and/or hinged, thereby facilitating the forming of a gap between the upper and lower guards at the user's front teeth as the user's tongue thrusts forward. Whether or not the upper guard 26 and lower guard 28 are connected, the user's upper molars 18 and lower molars 22 serve to anchor the guards to the user's teeth. Thus, the oral appliance of the present disclosure can be worn by users who may grind their teeth while they sleep while achieving its intended function and purpose.

In some embodiments, the upper and lower guards may be constructed of any suitable biocompatible or orally-compatible materials, including, for example, polymers, or acrylics. Other suitable materials include thermoplastic polymers, thermosets, thermoplastic elastomers, all of which should be capable of retaining their shape when worn by the subject and thus subjected to temperature of at least 98.6F. or higher. Suitable biocompatible or orally-compatible materials useful for the construction oral appliances to be worn in a user's mouth are well known to one of ordinary skill in the art.

In some embodiments, the oral appliance as described above can further comprise a means for immobilizing the tongue in an extended position between the user's upper and lower front teeth and away from their lingual surfaces. The means for immobilizing can include any mechanism or structure capable of retaining the tongue in an extended position between the upper and lower front teeth and away from their lingual surfaces. In some embodiments, the means for immobilizing can comprise an adjustable clamping mechanism that provides sufficient pressure on the tongue to maintain it in a forward position for an extended period, for example during sleeping. It is contemplated that the means for immobilizing (such as an adjustable clamping mechanism) is attached to the upper guard and/or lower guard and is adjustable by the user once after placement of the tongue in the desired extended position. In some embodiments, the oral appliance comprising a means for immobilizing the tongue in an extended position between the upper and lower front teeth and away from their lingual surfaces can be worn by a user during sleeping and thereby reduce or prevent sleep apnea, or other sleep disorders resulting from improper tongue positioning.

As described elsewhere herein, the oral appliance of the present disclosure is designed to be worn inside a user's mouth 12 as illustrated in FIGS. 5 and 10. The design of the oral appliance allows at least the user's upper molars 18 and lower molars 22 serve to anchor the upper and lower guards to the user's teeth. The design of the oral appliance allows a user to easily insert and position the upper and lower guards (either separately or as a single piece) into his or her own mouth such that the user's teeth fit into the recesses located on the upper surface of the upper guard and lower surface of the lower guard. Upon insertion of the oral appliance into the user's mouth, the lower surface of the user's tongue 24 becomes positioned on the ascending ramp 37 of the upper surface 54 of the lower guard 28. As described elsewhere herein, because the lower surface of the upper guard and upper surface of the lower guard are devoid of any surfaces against which the user's tongue can thrust and/or which can prevent a user's tongue from extending forward, the oral appliance when worn in the user's mouth allows the tongue to extend forward, including extending through a gap between the user's front teeth. As described elsewhere herein, the design of the upper and lower guards facilitates the formation of a gap between the user's teeth as the tongue extends. As illustrated in FIG. 11, by allowing the tongue to extend forward when worn in the mouth, the oral appliance also facilitates the opening of the user's airway 25 more than it opens without the oral appliance present. Accordingly, the use of the oral appliance not only reduces or prevents tongue thrusting against a lingual surface but also facilitates forward tongue positioning that results in increased opening of the airway 25. Accordingly, use of an oral appliance of the present disclosure can provide a therapeutic treatment for reducing or preventing disorders associated with improper tongue positioning, such as tongue thrusting or sleep disorders (e.g., sleep apnea).

In some embodiments, the present disclosure provides a use or method for treating improper tongue positioning in a subject, wherein the use or method comprises the subject wearing an oral appliance of the present disclosure in the mouth. In some embodiments, the subject wears the oral appliance in the mouth during periods of time when the subject is susceptible to improper tongue positioning, such as, while the subject is sleeping.

As described elsewhere herein, the uses or methods of treatment in which a subject wears the oral appliance in the mouth causes the tongue to adopt and/or maintain a more forward extended position than it can adopt and/or maintain without the oral appliance present. In some embodiments, the use of the oral appliance in a method of treatment allows the tongue to adopt and/or maintain an extended forward position between the upper and lower front teeth (see e.g., FIG. 11), and even adopt a position with the tongue's tip extended between the lips and out of the mouth.

In some embodiments, the uses of the oral appliance and/or methods of treatment, comprises a use and/or method for treating or preventing a sleep disorder. In such embodiments, the use and/or method comprises positioning an oral appliance of the present disclosure in a subject's mouth during sleep to adopt and/or maintain a user's tongue in an extended position. In some embodiments, the extended position of the user's tongue comprises a position between the upper and lower front teeth and away from their lingual surfaces.

Without limiting the foregoing description, certain non-limiting clauses of the disclosure numbered as below are provided, wherein each of the individually numbered clauses may be used or combined with any of the preceding or following clauses. Thus, these clauses are intended to provide support for all such combinations and is not necessarily limited to specific combinations explicitly provided below:

1. An oral appliance comprising an upper guard and a lower guard, wherein the upper guard has a lower surface that is ramped with a descending slope from a posterior end of the upper guard to an anterior end of the upper guard; and wherein the lower guard has an upper surface that is ramped with an ascending slope from a posterior end of the lower guard to an anterior end of the lower guard.

2. The oral appliance of clause 1, wherein the lower surface of the upper guard is devoid of any lingual surface that is substantially vertical; and the upper surface of the lower guard that is devoid of any lingual surface that is substantially vertical.

3. The oral appliance of any one of clauses 1-2, wherein the lower surface of the upper guard is devoid of any surface that is substantially parallel to a lingual surface of a user's front upper teeth; and the upper surface of the lower guard is devoid of any surface that is substantially parallel to a lingual surface of a user's front lower teeth.

4. The oral appliance of any one of clauses 1-3, wherein the descending and ascending ramped configuration of the upper and lower guards acts to guide a user's tongue away from their lingual surfaces.

5. The oral appliance of any one of clauses 1-4, wherein the descending and ascending ramped configuration of the upper and lower guards acts to guide a user's tongue to extend between the upper and lower front teeth.

6. The oral appliance of any one of clauses 1-5, wherein the upper guard and the lower guard comprise risers between the user's molars that keep a user's teeth from fully occluding, thereby forming a gap between the user's upper and lower front teeth.

7. The oral appliance any one of clauses 1-6, wherein the ascending ramp of the lower guard and the descending ramp of the upper guard direct the user's tongue tip when thrust forward to protrude from the gap between the upper and lower front teeth.

8. The oral appliance of any one of clauses 1-7, further comprising a means for immobilizing the tongue in an extended position between the upper and lower front teeth.

9. The oral appliance of any one of clauses 1-8, wherein the upper guard has a first posterior end, a first anterior end, a first upper surface, a first lower surface, and a first recess formed in the first upper surface configured to receive a user's upper teeth; and the lower guard has a second posterior end, a second anterior end, a second upper surface, a second lower surface, and a second recess formed in the second lower surface configured to receive the user's lower teeth.

10. The oral appliance of clause 9, wherein the first lower surface of the upper guard is configured without a lingual surface substantially parallel to a lingual surface of the first recess, and the second upper surface of the lower guard is configured without a lingual surface substantially parallel to a lingual surface of the second recess, whereby the oral appliance provides no surface against which the user's tongue can thrust.

11. The oral appliance of clause 9, wherein the first lower surface of the upper guard has a substantially continuous descending slope from the first posterior end to the first anterior end; and the second upper surface of the lower guard has a substantially continuous ascending slope from the second posterior end to the second anterior end.

12. The oral appliance of clause 11, wherein the descending first lower surface of the upper guard and the ascending second upper surface of the lower guard are configured to guide a user's tongue toward a gap between anterior ends of the upper and lower guards.

13. The oral appliance of clause 9, wherein the first lower surface of the upper guard below a portion of the first curved recess configured to receive the user's upper molars has a riser configured to prevent full occlusion by the user.

14. The oral appliance of clause 9, wherein the second upper surface of the lower guard above a portion of the second curved recess configured to receive the user's lower molars has a riser to prevent full occlusion by the user.

15. The oral appliance of clause 9, wherein the upper guard and the lower guard are detached from each other.

16. The oral appliance of clause 9, wherein the upper guard and the lower guard have one or more hinge connections at their respective posterior ends.

17. A method for treating or preventing disorders associated with improper tongue positioning, wherein the method comprises using an oral appliance of any one of claims 1 to 16, whereby a user's tongue extends in a forward position between the upper and lower front teeth and away from their lingual surfaces.

18. The method of clause 17, wherein the disorder is tongue thrusting.

19. The method of clause 17, wherein the disorder is a sleep disorder; optionally, wherein the sleep disorder is sleep apnea.

20. The method of any one of clauses 17-19, wherein the method further comprises using the oral appliance during sleep.

21. An oral appliance of any one of clauses 1 to 16 for use in a method of treating the human body by therapy.

22. An oral appliance of any one of clauses 1 to 16 for use in a method of treating a disorder associated with improper tongue positioning in a human; optionally, wherein the disorder is tongue-thrusting or a sleep-disorder.

23. The oral appliance of clause 22, wherein the disorder is sleep apnea.

In the preceding description, for the purposes of explanation, numerous specific details are set forth to provide a thorough understanding of various embodiments. It is apparent, however, that various embodiments may be practiced without these specific details or with one or more equivalent arrangements. In other instances, well-known structures and devices are shown in block diagram form in order to avoid unnecessarily obscuring various embodiments.

The terminology used herein is for the purpose of describing particular embodiments only and is not intended to be limiting of the present disclosure. As used herein, the term “substantially,” “about,” “approximately,” and similar terms are used as terms of approximation and not as terms of degree, and are intended to account for the inherent deviations in measured or calculated values that would be recognized by those of ordinary skill in the art. “About” or “approximately,” as used herein, is inclusive of the stated value and means within an acceptable range of deviation for the particular value as determined by one of ordinary skill in the art, considering the measurement in question and the error associated with measurement of the particular quantity (i.e., the limitations of the measurement system).

For the descriptions herein and the appended claims, the singular forms “a”, and “an” include plural referents unless the context clearly indicates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. The use of “comprise,” “comprises,” “comprising” “include,” “includes,” and “including” are interchangeable and not intended to be limiting. It is to be further understood that where descriptions of various embodiments use the term “comprising,” those skilled in the art would understand that in some specific instances, an embodiment can be alternatively described using language “consisting essentially of” or “consisting of.”

Generally, the nomenclature used herein, and the techniques and procedures described herein include those that are well understood and commonly employed by those of ordinary skill in the art. Unless defined otherwise, all technical and scientific terms used herein have the same meaning as is commonly understood by one of ordinary skill in the art to which this disclosure pertains.

Various embodiments are described herein with reference to sectional illustrations that are schematic illustrations of embodiments and/or intermediate structures. As such, variations from the shapes of the illustrations as a result, for example, of manufacturing techniques and/or tolerances, are to be expected. Further, specific structural or functional descriptions disclosed herein are merely illustrative for the purpose of describing embodiments according to the concept of the present disclosure. Thus, embodiments disclosed herein should not be construed as limited to the particular illustrated shapes of regions, but are to include deviations in shapes that result from, for instance, manufacturing. Thus, the regions illustrated in the drawings are schematic in nature and their shapes are not intended to illustrate the actual shape of a region of a device and are not intended to be limiting.

Additional embodiments of the invention are set forth in the following claims. It will be clear to one skilled in the art that various modifications or changes to the examples, descriptions, and embodiments described herein can be made and are to be included within the breadth of the appended claims. Further, one of skill in the art will recognize a number of equivalent structures and methods to those described herein. All such equivalents are to be understood to be within the scope of the present disclosure and are covered by the appended claims.

The disclosures of all publications, patent applications, patents, or other documents mentioned herein are expressly incorporated by reference in their entirety for all purposes to the same extent as if each such individual publication, patent, patent application or other document were individually specifically indicated to be incorporated by reference herein in its entirety for all purposes and were set forth in its entirety herein. In case of conflict, the present specification, including specified terms, will control.

1. An oral appliance comprising an upper guard and a lower guard, wherein the upper guard has a lower surface that is ramped with a descending slope from a posterior end of the upper guard to an anterior end of the upper guard; and wherein the lower guard has an upper surface that is ramped with an ascending slope from a posterior end of the lower guard to an anterior end of the lower guard.

2. The oral appliance of claim 1, wherein the lower surface of the upper guard is devoid of any lingual surface that is substantially vertical; and the upper surface of the lower guard that is devoid of any lingual surface that is substantially vertical.

3. The oral appliance of claim 1, wherein the lower surface of the upper guard is devoid of any surface that is substantially parallel to a lingual surface of a user's front upper teeth; and the upper surface of the lower guard is devoid of any surface that is substantially parallel to a lingual surface of a user's front lower teeth.

4. The oral appliance of claim 1, wherein the descending and ascending ramped configuration of the upper and lower guards acts to guide a user's tongue away from their lingual surfaces.

5. The oral appliance of claim 1, wherein the descending and ascending ramped configuration of the upper and lower guards acts to guide a user's tongue to extend between the upper and lower front teeth.

6. The oral appliance of claim 1, wherein the upper guard and/or the lower guard comprise risers located above the user's molars that keep a user's teeth from fully occluding, thereby forming a gap between the user's upper and lower front teeth.

7. The oral appliance of claim 1, wherein the ascending ramp of the lower guard and the descending ramp of the upper guard direct the user's tongue tip when thrust forward to protrude from a gap between the upper and lower front teeth.

8. The oral appliance of claim 1, further comprising a means for immobilizing the tongue in an extended position between the upper and lower front teeth.

9. The oral appliance of claim 1, wherein the upper guard and the lower guard are detached from each other.

10. The oral appliance of claim 1, wherein the upper guard and the lower guard are attached at one or more locations at their respective posterior ends.

11. The oral appliance of claim 10, wherein the attachment locations are flexible and/or hinged.

12. The oral appliance of claim 1, wherein the upper guard has a recess formed in the upper surface configured to receive a user's upper teeth; and the lower guard has a recess formed in the lower surface configured to receive the user's lower teeth.

13. The oral appliance of claim 12, wherein the lower surface of the upper guard is configured without a surface substantially parallel to a lingual surface of the recess configured to receive a user's upper teeth, and the upper surface of the lower guard is configured without a surface substantially parallel to a lingual surface of the recess configured to receive a user's lower teeth, whereby the oral appliance provides no surface against which the user's tongue can thrust.

14. The oral appliance of claim 12, wherein the lower surface of the upper guard below a portion of the curved recess configured to receive the user's upper molars has a riser configured to prevent full occlusion by the user; and/or the upper surface of the lower guard above a portion of the curved recess configured to receive the user's lower molars has a riser to prevent full occlusion by the user.

15. A method for treating or preventing disorders associated with improper tongue positioning, wherein the method comprises using an oral appliance of any one of claim 1, whereby a user's tongue extends in a forward position between the upper and lower front teeth and away from their lingual surfaces.

16. The method of claim 15, wherein the method further comprises using the oral appliance during sleep.

17. The method of claim 15, wherein the disorder is tongue thrusting.

18. The method of claim 15, wherein the disorder is a sleep disorder.

19. The method of claim 15, wherein the disorder is sleep apnea.
